(19) United States
(12) Patent Application Publication
(10) Pub. No.: US 2008/0041750 A1
(43) Pub. Date: Feb. 21, 2008

(54) Process for making packages for hygiene articles and packages made therefrom

(76) Inventor: Christian Kohlweyer, Bad Vilbel (DE)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/811,036

(22) Filed: Jun. 8, 2007

(30) Foreign Application Priority Data
Jun. 29, 2006 (EP) EP 06116316.8

(51) Int. Cl. A61B 19/02

(52) U.S. Cl. 206/438; 53/412

(57) ABSTRACT

A package for hygiene articles having an opening flap, which is fastened to the package by an adhesive strip. The adhesive strip is located offset from the centerline of the opening flap.

### FIELD OF THE INVENTION

The present invention relates to a package for hygiene articles. The package has an opening flap which is fastened to the package by an adhesive strip. The adhesive strip is located offset from the centerline of the opening flap.

### BACKGROUND OF THE INVENTION

Packages made of film material for storing and dispensing hygiene articles are widely known in the art and are marketed from many manufacturers for many different products. Examples are plastic film packages containing a stack of sanitary napkins, such as those marketed by The Procter & Gamble Company under the trade name ALWAYS®.

Many of these film packages have opening flaps with a reclosing functionality, which oftentimes is provided by a perforation sized and shaped for providing a flap when opened, and a small adhesive strip, which is fixedly attached to the free end of the opening flap and which can releasably seal the free end of the flap to the package material. When using conventional processes of making the package, such as the wicket bag process, the adhesive strip is typically attached to the opening flap during or after the package is formed and filled with its contents.

It would be desirable to provide a simplified process which allows making the complete package prior to filling, wherein the adhesive strip and the perforation which creates the opening flap are already incorporated into the pre-made package, and fully integrated into the existing bag making process.

### SUMMARY OF THE INVENTION

The present inventors have addressed the above need by providing a process in that the adhesive strip is displaced out of any fold lines. This method allows incorporation of the adhesive strip into the package already on the production line of the package prior to filling it with contents. The process comprises the following steps:
A) supplying a package material in a machine direction,
B) providing the package material with means for providing an opening flap, the means are selected from perforations or weakness lines,
C) applying an adhesive strip to the package material, bridging the perforations or weakness lines,
D) folding the perforated package material by applying fold lines extending in machine direction, such that at least one inner fold line extends between two outer fold lines such that the package material assumes a substantially W-shaped cross section with the adhesive strip located outside the fold lines created,
E) cut-sealing the continuous package material in cross direction CD, whereby forming individual compartments.

### BRIEF DESCRIPTION OF THE DRAWINGS

FIG. 1 provides an overview over the process of the present invention.

FIG. 2 illustrates the joining of the adhesive strip to the package material, bridging the perforations or weakness line.

FIGS. 3 and 4 illustrate the folding of the package material subsequent to the joining of the adhesive strip.

FIG. 5 illustrates the package of the present invention with the still open bottom oriented upwards.

FIGS. 6 and 7 illustrate the package of the present invention with a stack of hygiene articles in its interior, wherein FIG. 6 shows the still open compartment and FIG. 7 shows the closed package.

### DETAILED DESCRIPTION OF THE INVENTION

“Hygiene articles” as used herein refers to products for personal hygienic care, including disposable articles. Typical disposable hygiene products include infant diapers, sanitary napkins, panty liners, breast pads, tampons and the like.

“Package” as used herein refers to a material provided to surround or enclose hygiene products. The package according to the present invention is typically made from polymeric film like polyethylene (PE), polypropylene (PP), laminates, woven webs or fabrics. Polymeric films also include blown or cast film materials in a blend of low density polyethylene (LDPE), linear low density polyethylene (LLDPE), metallocene PE blends (metPE), ethylene vinyl acetate, Surlyn®, polyethylene terephthalate (PET), mono- and biaxially oriented polypropylene (M/BoPP) and nylon. Woven and non-woven webs can be formed from monocomponent fibres, bicomponent fibres, multiconstituent fibres, capillary channel fibres and the like. A polymeric film can be two or more films laminated together. A polymeric film can be pigmented. A polymeric film can be clear or opaque. The package may enclose the hygiene articles in a hygienically protective manner. The hygiene articles are typically arranged in one or more stacks inside the package. The package herein is provided with an opening flap for allowing a user to access and dispense the hygiene articles therethrough. The opening is typically created by a perforation in the package material, which, when torn apart, provides an opening in the package material and an opening flap. The opening flap can be reclosed by use of an adhesive strip which is fixedly joined to the opening flap and which is capable of releasable attachment to the package material.

“Machine direction” as used herein refers to the direction of the production line. “Cross-direction” as used herein refers to a direction, which extends perpendicularly to said machine direction.

“Perforation” as used herein refers to a true hole in the package material. “Weakness line” as used herein refers to areas in which the package material has a reduced tear resistance compared to regions outside the weakness line. The weakness line can be provided by continuous lines or by a line of weakness points. The reduction of the tear force can be achieved by any suitable means known in the art, such as reducing the thickness of the package material by e.g. cutting, punching, mechanical or ultrasonic embossing or by changing the crystalline structure of the package material by e.g. applying heat, a laser beam, high voltage or the like.

“Centerline” as used herein refers to the longitudinal axis of symmetry of a surface. For instance, the centerline of an opening flap is the axis of symmetry of that flap, which crosses its line of permanent jointure to the package body.

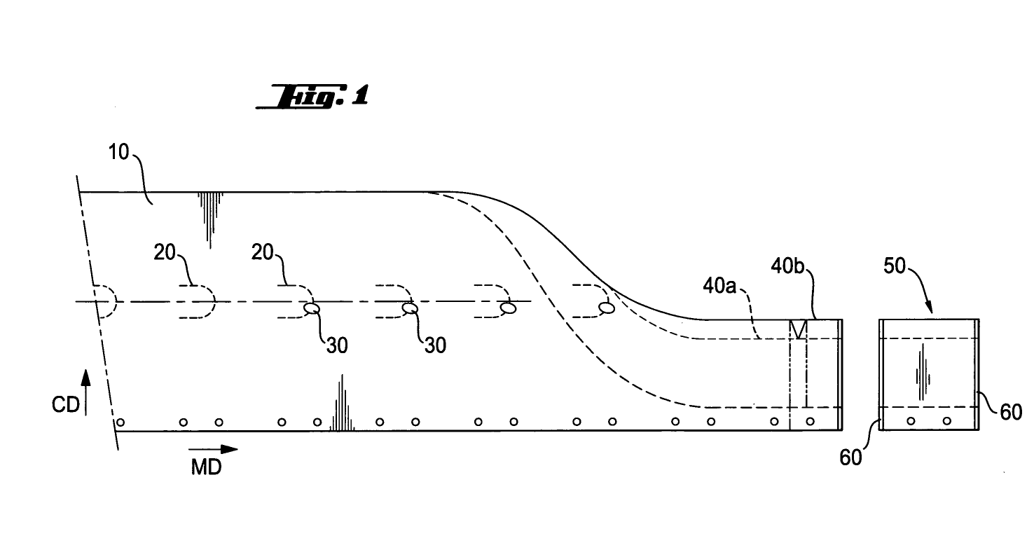

The process of the present invention is illustrated in FIG. 1 and can be structured in a sequence of individual steps which need not be in any particular order.

In one step, the package material (10) is supplied in a machine direction MD.

In another step the package material (10) is provided with the means for providing the opening flap (20). Suitable means (20) include perforations or weakness lines. Perforations can be provided by a perforation means, such as a cutting or punching diehead or a perforation roller and weakness lines can be provided e.g. by applying heat. The perforations or weakness lines can have any size and shape, which is suitable for providing a reclosable dispensing opening in the package formed. Exemplary shapes are semicircles, semiellipsoids, wedges or open squares or rectangles.

In another step, an adhesive strip (30) is applied to the perforated or weakened package material (10). The adhesive strip (30) is arranged such that it bridges the perforations or weakness lines created in step B). The adhesive strip (30) is sized and shaped for providing a user graspable means for opening the perforations or weakness lines. Suitable shapes of the adhesive strip (30) are circular shapes, square shapes or oblong shapes, such as ellipsoidal, rectangular, drop-shaped, dogbone shaped or irregular shapes. The adhesive strip (30) is fixedly joined to the side of the perforations or weakness lines providing the opening and releasable joined to the side of the package material which will be surrounding the opening once the perforations or weakness lines are torn off. This can be achieved by using different adhesives or other suitable means known in the art. Due to its arrangement outside the fold lines, the adhesive strip (30) will typically be offset from the centerline of the perforations or weakness lines.

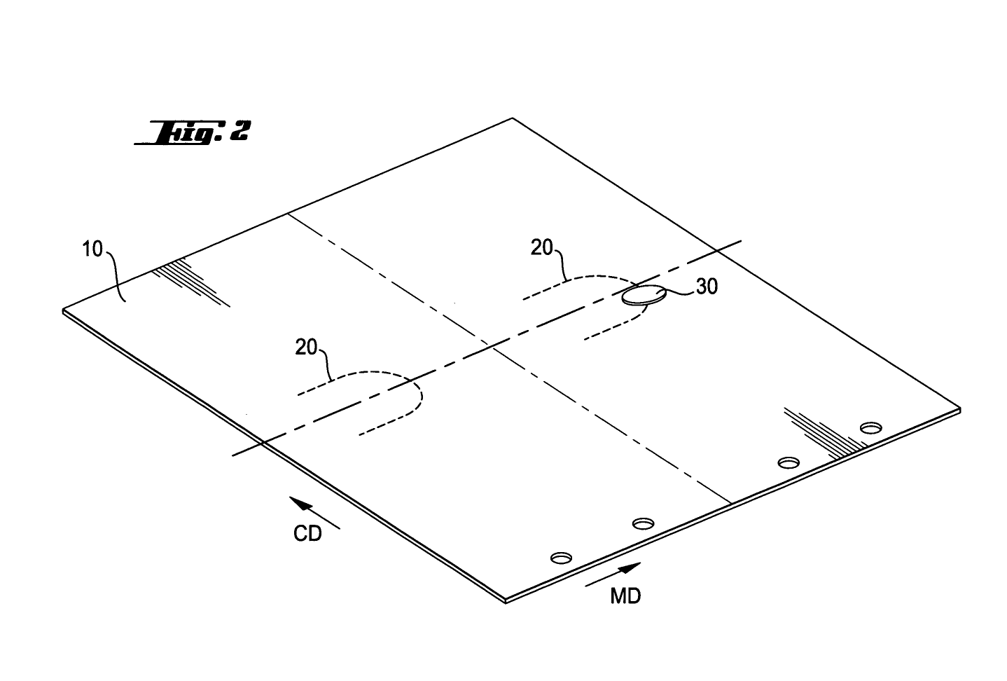

The present inventors have found that operating oblong adhesive strips (30) for opening the flap can be improved if the oblong adhesive strip is distorted versus the machine direction. It has been found that a distortion by an angle of from about 0° to about 360°, from about 0° to about 90°, from about 0° to about 45°, from about 10° to about 20° and for some applications about 15° versus MD are beneficial in this context. Such distortion can be achieved by applying the adhesive strip (30) by a roll rotating in MD but being supplied with the adhesive strips in distorted orientation already. FIG. 2 provides an exemplary overview of the application of the adhesive strip (30) according to the present invention.

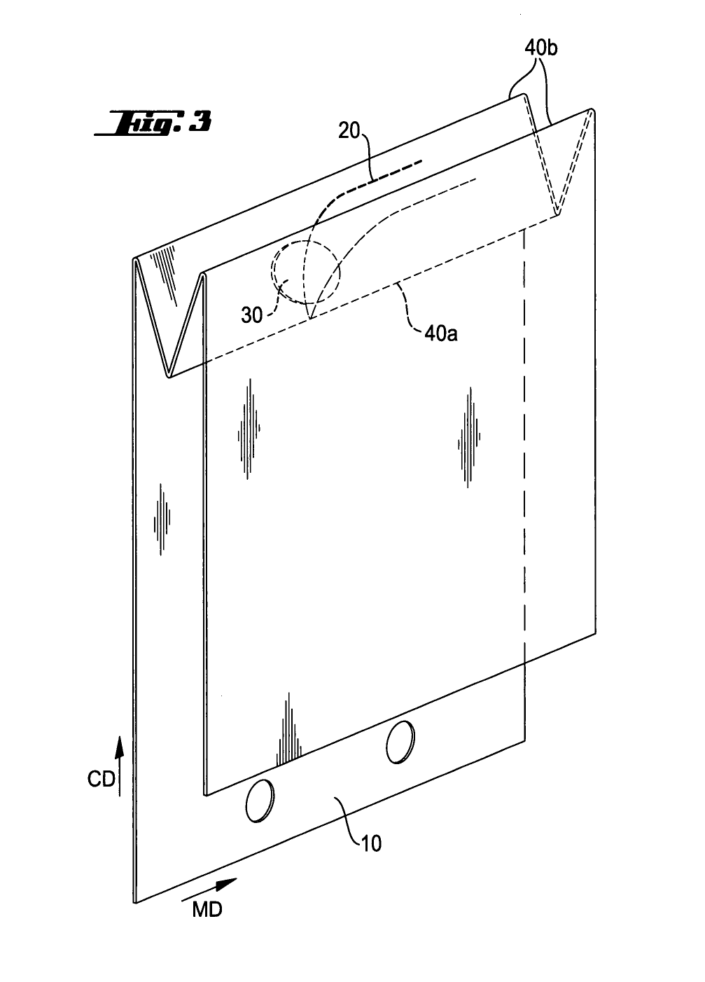

In another step, the perforated or weakened package material (10) is folded by applying fold lines substantially extending in machine direction, such that at least one inner fold line (40a) extends between two outer fold lines (40b) such that the package material assumes a substantially W-shaped cross section. The fold lines (40a,b) are arranged such that the adhesive strip (30) is located outside the fold lines (40a,b). FIG. 3 illustrates a typical manner of folding of the package material (10) according to the process of the present invention.

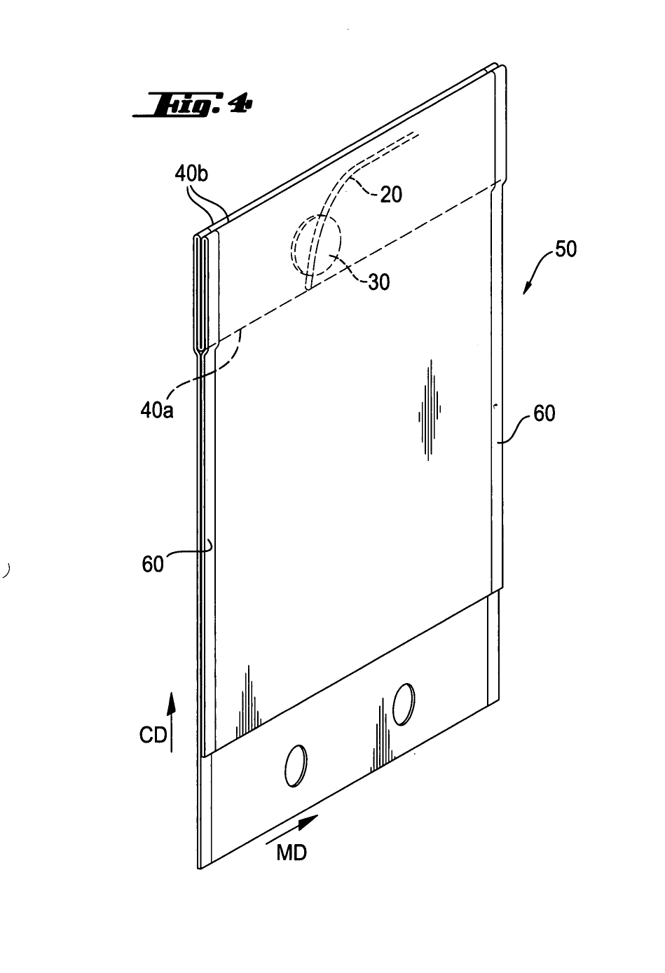

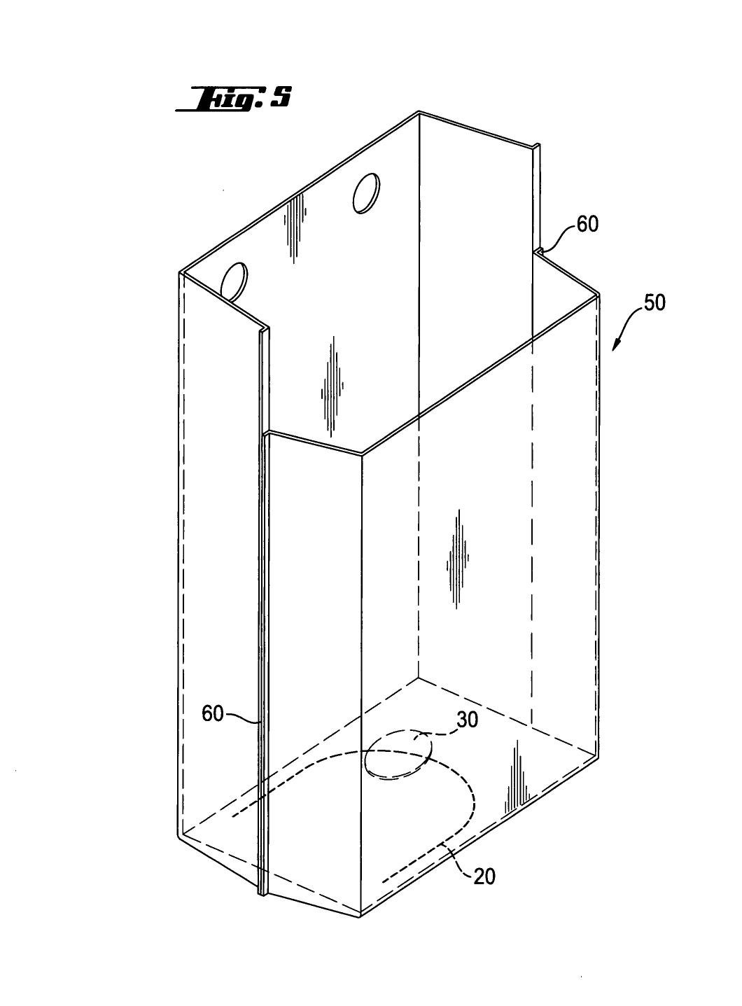

In another step, the folded package material (10) is cut-sealed in cross direction such that the cuts do not bridge the perforations or weakness lines. Suitable means for cut-sealing are heat cutting, pressure cutting, induction sealing, ultrasonic bonding and the like. The cut-sealing results in separating the package material into individual compartments (50), which are still open on one side but are sealed in CD by seal lines (60). FIGS. 4 and 5 show typical compartments (50) made by the process of the present invention.

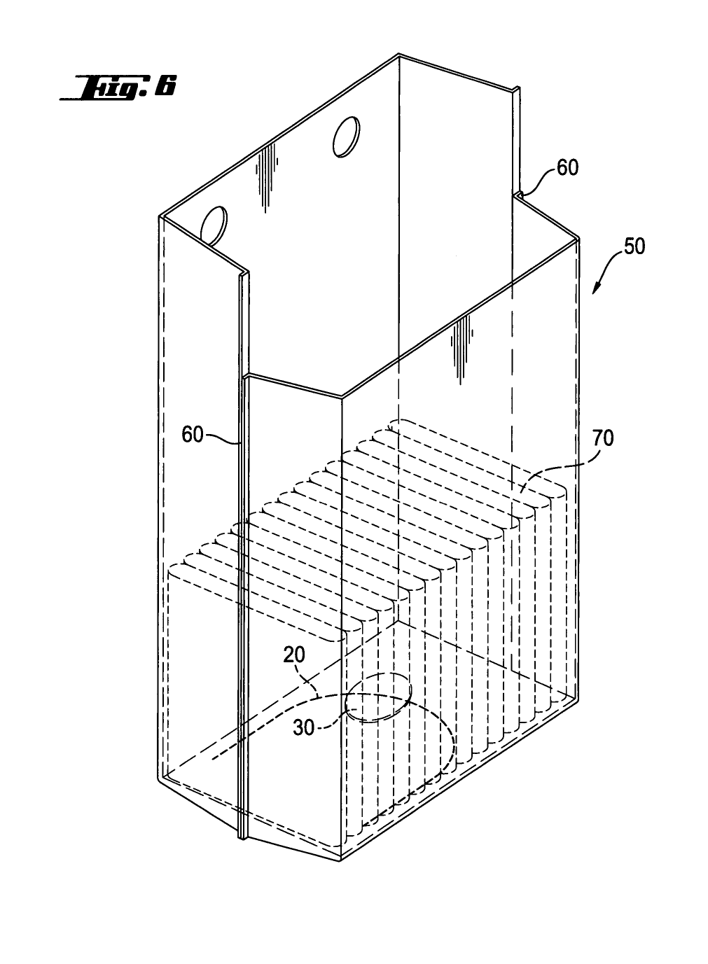

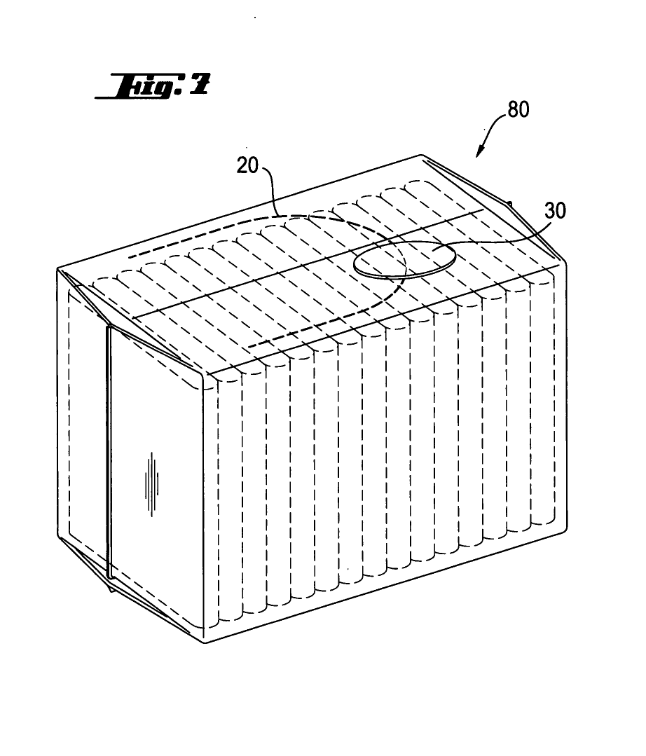

As shown in FIGS. 6 and 7 the compartments (50) are suitable for being used in a so-called wicket bag making process by feeding them into a magazine of a filling line, which takes the compartments (50) out of the magazine, arranges them to assume a 3-dimensional configuration as illustrated in FIG. 5, such as a box-like shape, and fills them with contents, such as at least one stack of hygiene articles (70), through the open side of the compartment (50), as illustrated in FIG. 6. After the filling step the open side of compartments (50) is closed to form a package (80), which fully encloses the articles (70) stored therein.

In a second aspect, the present invention relates to a package (80) for hygiene articles (70) which is provided with a reclosable opening flap, wherein the reclosing functionality is provided by an adhesive strip (30) which is arranged offset with respect to the centerline of the flap. The package (80) is sized and shaped for accommodating hygiene articles (70), which are typically arranged in one or more stacks. Suitable shapes are parallelepipedal, roll-like and the like.

The reclosable opening flap can be provided by any suitable means known therefore, such as perforations or weakness lines, which, when torn off, provide the opening flap. The opening flap is sized and shaped for providing a sufficiently large dispensing opening for allowing convenient dispensing of the hygiene articles (70) stored in the package (80).

The adhesive strip (30) can have any suitable size and shape providing for sufficient bridging of the perforations or weakness lines providing the opening flap for allowing secure reclosing. Suitable shapes are circular shapes, square shapes or oblong shapes, such as ellipsoidal, rectangular, drop-shaped, dogbone shaped or irregular shapes. The adhesive strip (30) is fixedly joined to the side of the perforations or weakness lines providing the dispensing opening and releasably joined to the side of the package material which will be surrounding the dispensing opening once the perforations or weakness lines are torn off. This can be achieved by using different adhesives or other suitable means known in the art.

The adhesive strip (30) is arranged offset of the centerline of the opening flap and/or outside of any fold line in the package material. This has at least two benefits. First of all, the centerline of the opening flap is in many cases coextensive with the inner fold line (40a) of the package material (10), which results from the process of making the package (80). This fold line (40a), although substantially flattened, is in many cases still present on the package (80) and thus may not provide a desired basis for attachment of the adhesive strip (30) because the package material (10) is not completely flat in the region of the fold line (40a). Further, an adhesive strip (30) located offset of the centerline of the flap can be grasped and operated more conveniently as it is arranged more ergonomically when considering human anatomy.

The present inventors have found that operating oblong adhesive strips (30) for opening the flap of the package (80) can be improved if the oblong adhesive strip (30) is distorted versus the centerline of the flap and thus, the centerline of the perforations providing the flap, and/or distorted with respect to the centerline of the surface of the package being provided with the perforation or weakness line. It has been found that a distortion by an angle of from about 0° to about 360°, from about 0° to about 90°, from about 0° to about 45°, from about 10° to about 20° and for some applications 15° versus the centerline of the opening flap and/or distorted with respect to the centerline of the surface of the package being provided with the perforation or weakness line are beneficial in this context. The adhesive strip (30) can be coloured and/or provided with indicia, such as letters, pictograms, graphics or other information. By this the adhesive strip (30) can be used for conveying information to the consumer such as size, absorbency or number of the hygiene articles stored in the package (80).

The distortion of the adhesive strip (30) is beneficial for keeping the adhesive strip off a fold line (40a,b) required by the wicket bag making process for laying flat the pre-made package (80). Further, the offset and distortion of the adhesive strip (30) is beneficial for opening of the package (80) because of human anatomy. When holding the package (80) for first time opening, the user will rather pull the adhesive strip (30) non-parallel with respect to the centerline of the perforation. By placing the adhesive strip (30) offset of the centerline, the adhesive strip (30) is arranged more ergonomically and opening the package (80) for dispensing the articles (70) contained therein is thus more convenient. That same distortion is also beneficial for integrating the strip application process into an existing wicket bag making process, in a way that it maintains the output, efficiency, consistency and/or quality of the process.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for making a package, the process comprising the following steps:
a) supplying a package material in a machine direction;
b) providing the package material with means for providing an opening flap, the means selected from perforations or weakness lines;
c) applying an adhesive strip to the package material bridging the perforations or weakness lines;
d) folding the perforated package material by applying fold lines extending in the machine direction, such that at least one inner fold line extends between two outer fold lines such that the package material assumes a substantially W-shaped cross section with the adhesive strip located outside the fold lines created; and
e) cut-sealing the continuous package material in cross direction to form individual compartments.

2. The process of claim 1, wherein the perforations are provided by cutting die heads, punching die heads or perforation rollers.

3. The process of claim 1, wherein the weakness lines are provided by laser etching, ultrasound or heated plates.

4. The process of claim 1, wherein the perforations or weakness lines have a shape selected from semicircles, semiellipsoids, wedges, open squares or rectangles.

5. The process of claim 1, wherein the adhesive strip is fixedly joined to the side of the perforations or weakness lines providing the opening and releasable joined to the side of the package material which will be surrounding the opening once the perforations or weakness lines are torn off.

6. The process of claim 1, wherein the adhesive strip is placed distorted with respect to the machine direction by an angle of from about 0° to about 360°.

7. The process of claim 6 wherein said angle is from about 0° to about 90°.

8. The process of claim 6, wherein the adhesive strip is applied to the package material by rollers rotating in machine direction, which are provided with the adhesive strips in distorted orientation.

9. The process of claim 1, wherein step e) is facilitated by heat cutting or pressure cutting.

10. A package for housing hygiene articles, wherein the package is made according to claim 1.

11. A package made of a package material, the package containing one or more hygiene articles, the package comprising:
a reclosable opening flap for providing a dispensing opening, the opening flap being provided with means for providing an opening flap, the means selected from perforations or weakness lines; and
an adhesive strip bridging the perforations or weakness lines and being sized and shaped for providing secure reclosing, the adhesive strip is fixedly joined to the side of the perforations or weakness lines providing the opening flap and releasably joined to the package material on the side of the perforations which is surrounding the dispensing opening once the perforations or weakness lines are torn off, wherein the adhesive strip is arranged offset from the centerline of the perforation or weakness line providing the opening flap.

12. The package of claim 11, wherein the adhesive strip is arranged outside of any fold lines being present in the package material.

13. The package of claim 11, wherein the adhesive strip has an oblong shape.

14. The package of claim 13, wherein the adhesive strip is arranged distorted with respect to the centerline of the perforation or weakness line providing the opening flap and/or is distorted with respect to the centerline of the surface of the package being provided with the perforation or weakness line by an angle of from about 0° to about 360°.

15. The package of claim 14 wherein the angle is from about 0° to about 90°.

16. The package of any of claim 11, wherein the adhesive strip is provided with color and/or indicia.
